(19) United States
(12) Patent Application Publication
(10) Pub. No.: US 2023/0293241 A1
(43) Pub. Date: Sep. 21, 2023

(54) METHOD FOR PROVIDING SURGICAL IMPLANT

(71) Applicant: ANYMEDI INC., Seoul (KR)

(72) Inventors: Gukbae KIM, Seoul (KR); Seunghyun CHOI, Seoul (KR); Hyunwe PARK, Incheon (KR)

(21) Appl. No.: 18/201,576

(22) Filed: May 24, 2023

Related U.S. Application Data
(63) Continuation of application No. PCT/KR2021/017351, filed on Nov. 24, 2021.

(30) Foreign Application Priority Data
Nov. 24, 2020 (KR) 10-2020-0158797

(51) Int. Cl. A61B 34/10; A61F 2/00; A61F 2/18

(52) U.S. Cl. CPC A61B 34/10; A61F 2/0059; A61F 2/186; A61B 2034/108; A61B 2034/104; A61B 2034/105; A61F 2240/002

(57) ABSTRACT

A method for providing a surgical implant, based on a system for a first terminal for initially designing a surgical plan by a patient, a second terminal for finalizing a surgical plan by a surgeon, and a server for communicating with the first terminal and the second terminal and further designing an implant according to the surgical plan, including, obtaining, by the server, a first 3D image based on a patient medical image data including at least one of a surgical part; transmitting, by the server, the first 3D image to the first terminal to obtain at least one modified second 3D image corresponding to a surgical plan initially designed by the patient, from the first terminal; transmitting, by the server, the at least one modified second 3D image to the second terminal to obtain a final third 3D image corresponding to a surgical plan designed by the surgeon, from said second terminal; and designing, by said server, an implant based on the patient medical image data, the first 3D image, the second 3D image, and/or the third 3D image.

### CROSS-REFERENCE TO RELATED APPLICATION

This application is a continuation application of international patent application No. PCT/KR2021/017351, filed on Nov. 24, 2021, which claims priority to Korean patent application No. 10-2020-0158797, filed on Nov. 24, 2020, which are hereby incorporated by reference in their entirety.

### BACKGROUND OF THE INVENTION

### Field of the Invention

Embodiments relate to a method for providing a surgical implant, and more particularly, are applicable to a patient specific instrument technology by referring to all of a patient's suggestion and a surgeon's opinion for modelling a surgical implant.

### Discussion of the Related Art

In recent years, there has been an explosion of interest in cosmetic surgery, along with an increased focus on beauty. One of the most popular facial procedures is rhinoplasty, which involves inserting an artificial filler such as silicone implant inside the nose to heighten the external appearance of the nose.

In the past, rhinoplasty surgery planning was limited to drawing lines on a side view photo of the patient's face, and in recent years, only in the case of revision surgery, a patient's CT (especially cone-beam CT) was taken to determine the condition of the existing implant.

After implementing a surgical plan using a qualitative analysis, surgery was performed according to the rhinoplasty surgical plan by sculpting the ready-made implant directly by a surgeon during the operation in the operating room after resecting the internal tissue of the patient's nose, relying on the surgeon's experience and senses in the operating room.

However, the problem with this method is that it takes a lot of time to carve, and it is not easy to create the desired shape of the implant by hand carving, and furthermore, it can damage the patient's internal tissues, causing excessive postoperative swelling and other postoperative side effects.

Even in the case of 3D conversion of CT images for surgical planning, it is only limited to accessing the internal anatomy of the nose by evaluating the pre-inserted implant, so this method still has the disadvantage that the patient's opinion is not reflected at all, and the implant is made only by the surgeon's qualitative judgment.

### SUMMARY OF THE INVENTION

Accordingly, it is an object of the present disclosure to provide a method for manufacturing surgical implants that applies to a patient-specific instrument technology by using a patient's suggestion and a surgeon's opinion for modeling a surgical implant.

Another object of the present invention is to provide a method of fabricating surgical implants that does not require intraoperative carving of the implant.

Accordingly, the present disclosure is directed to a method for providing surgical implants, which substantially obviates one or more problems due to limitations and disadvantages of the related art.

In addition to the objects of the present disclosure as mentioned above, additional objects and features of the present disclosure will be clearly understood by those skilled in the art from the following description of the present disclosure.

In accordance with an aspect of the disclosure, a method for fabricating a surgical implant is provided.

To achieve these objects and other advantages and in accordance with the purpose of the invention, as embodied and broadly described herein, a method for providing Surgical implants, based on a system for a first terminal for initially designing a surgical plan by a patient, a second terminal for finalizing a surgical plan by a surgeon, and a server for communicating with the first terminal and the second terminal and further designing an implant according to the surgical plan, may include: obtaining, by the server, a first 3D image based on a patient medical image data including at least one of a surgical part; transmitting, by the server, the first 3D image to the first terminal to obtain at least one modified second 3D image corresponding to a surgical plan initially designed by the patient, from the first terminal; transmitting, by the server, the at least one modified second 3D image to the second terminal to obtain a final third 3D image corresponding to a surgical plan designed by the surgeon, from said second terminal; and designing, by said server, an implant based on the patient medical image data, the first 3D image, the second 3D image, and/or the third 3D image.

In addition, a method for providing surgical implants may further include providing software capable of modifying the first 3D image to the first terminal, wherein the modified second 3D image is generated using the software, before or at the time of transmitting the first 3D image to the first terminal, by the server.

In addition, the designing, by said server, the implant based on the patient medical image data, the first 3D image, the second 3D image, and/or the third 3D image, may include acquiring a placing surface for the implant to install on from the patient medical image data, and determining a shape and height of the implant, based on a difference among the third 3D image, the patient medical image data, the first 3D image, and/or the second 3D image relative to the defined placing surface.

To achieve these objects and other advantages and in accordance with the purpose of the invention, as embodied and broadly described herein, a method for providing surgical implants may include manufacturing the designed implant, by the server, wherein the manufacturing the designed implant may be performed by at least one of 3D printing, cutting, injection, mold, and vacuum forming.

In another aspect of the present disclosure, a method for providing Surgical implants based on a system for a first terminal for initially designing a surgical plan by a patient, a second terminal for finalizing a surgical plan by a surgeon, and a server for communicating with the first terminal and the second terminal and further designing an implant according to the surgical plan, may include acquiring, by the first terminal, a first 3D image based on patient medical image data, including at least a surgical part; generating, by the first terminal, at least one modified second 3D image corresponding to a surgical plan initially designed by the patient based on the acquired first 3D image; transmitting, by the first terminal, the at least one modified second 3D image to the server and/or the second terminal; receiving, by the first terminal, a third 3D image that is a final 3D image corresponding to a surgical plan finalized by the surgeon based on the at least one modified second 3D image generated; and confirming, by the first terminal, the third 3D image to allow the second terminal to design an implant based on the patient medical image data, the first 3D image, the second 3D image, and/or the third 3D image.

In addition, a method for providing surgical implants of the confirming, by the first terminal, the third 3D image to allow the second terminal to design an implant based on the patient medical image data, the first 3D image, the second 3D image, and/or the third 3D image, may include requesting designing the implant to the server, by the second terminal.

According to the embodiments of the present disclosure, a method for providing surgical implants may further include transmitting a message for confirming the third 3D image to the second terminal and/or server, by the first terminal.

In accordance with another aspect of the disclosure, a method for providing surgical implants include transmitting, by the first terminal, the confirmed message for the third 3D image, and may further includes transmitting a patient's opinion based on comparing the second 3D image with the third 3D image.

According to the embodiments, a method for providing surgical implants may further include the step of being provided to the first terminal, with software capable of modifying the second 3D image, before the step of generating, by the first terminal, at least one modified second 3D image corresponding to a surgical plan initially designed by the patient based on the obtained first 3D image.

In addition, the modified second 3D image may be generated by at least one of a click-and-drag method and a numerical input method from a point at a particular location in a particular direction using the software.

In accordance with another aspect of the disclosure, a method for providing surgical implants based on a system for a first terminal for initially designing a surgical plan by a patient, a second terminal for finalizing a surgical plan by a surgeon, and a server for communicating with the first terminal and the second terminal and further designing an implant according to the surgical plan, the method may include: acquiring a 3D image, by the first terminal, based on a patient's medical image data including at least a surgical site; generating, by the first terminal, at least one modified 3D image corresponding to a surgical plan initially designed by the patient based on the acquired 3D image; acquiring, by the first terminal, a final 3D image corresponding to a surgical plan finalized by the surgeon based on the at least one modified 3D image; and confirming, by the first terminal, the final 3D image to allow the server to design an implant based on the patient medical image data, the 3D image, and the final 3D image.

In addition, a method for providing surgical implants may include the step of being provided, to the first terminal, with software capable of modifying said 3D image, prior to the step of generating at least one modified 3D image corresponding to a surgical plan initially designed by the patient based on the acquired 3D image, and the modified 3D image is generated using the software.

According to the embodiments, the modified 3D image is generated by at least one of a click-and-drag method and a numerical input method from a point at a particular location in a particular direction using the software.

The technical subjects pursued in embodiments of the disclosure may not be limited to the above mentioned technical subjects, and other technical subjects which are not mentioned may be clearly understood, through the following descriptions, by those skilled in the art to which the disclosure pertains.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a schematic of a system in which a method of fabricating a surgical implant may be practiced, according to an embodiment of the present disclosure;

FIG. 2 is a flow diagram illustrating an exemplary of a method for fabricating a surgical implant, according to an embodiment of the present disclosure;

FIG. 3 shows an example of a 3D image converted, according to an embodiment of the present disclosure;

FIGS. 4 through 6 are diagrams illustrating a process for modifying a 3D image in response to a patient initializing a surgical plan, according to an embodiment of the present disclosure;

FIGS. 7A to 8 are drawings illustrating a virtual surgery screens using a virtual surgery software, according to an embodiment of the present disclosure;

FIG. 9 illustrates a method for acquiring the height of an implant, according to an embodiment of the present disclosure;

FIG. 10 is a diagram illustrating the design process of an implant, according to an embodiment of the present disclosure;

FIG. 11 is a drawing illustrating the final design of an implant, according to an embodiment of the present disclosure;

FIG. 12 is a drawing illustrating an implant made, according to an embodiment of the present disclosure; and

FIGS. 13A and 13B are each flow diagrams of a method of fabricating a surgical implant, according to an embodiment of the present disclosure.

### DETAILED DESCRIPTION OF THE INVENTION

Hereinafter, exemplary embodiments of the present disclosure will be described in detail with reference to the accompanying drawings. However, the invention is not limited or defined by the exemplary embodiments. Identical reference numerals in each drawing designate members that perform substantially the same function.

Terms containing ordinal numbers, such as first, second, and the like, may be used to describe various components, but the components are not limited by such terms. These terms are used only to distinguish one component from another. For example, a first component may be named a second component, and similarly, a second component may be named a first component, without departing from the scope of the present disclosure. The terminology used in this application is merely used to describe specific embodiments and is not intended to limit the invention. Singular expressions include the plural unless the context clearly indicates otherwise.

The terms used in the present disclosure have been selected as widely used and generic as possible, taking into account their function in the present disclosure, but they may vary depending on the intention or precedent of those skilled in the art, the emergence of new technologies, etc. In addition, in certain cases, terms have been arbitrarily selected by the applicant, in which case their meaning will be described in detail in the description of the invention. Therefore, the terms used in the invention should be defined based on the meaning of the term and the overall content of the invention, and not simply on the name of the term.

Throughout the specification, when a part is said to “include” a component, this means that it may include additional components, not exclude other components, unless specifically stated to the contrary.

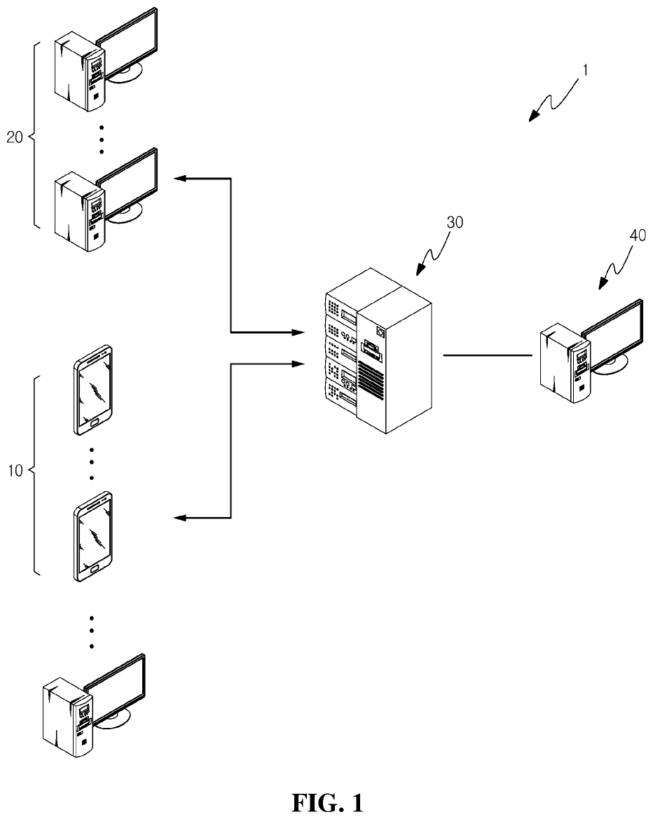

FIG. 1 is a block diagram of a system in which a method of fabricating a surgical implant according to the present disclosure may be practiced.

The system 1 includes a first terminal 10 for initially designing a surgical plan by a patient, a second terminal 20 for finalizing a surgical plan by a surgeon, and a server 30 for communicating with the first terminal 10 and the second terminal 20 and further designing a prosthesis according to the surgical plan.

The first terminal 10 is a computing device that can be used a patient to initially design a surgical plan, which can be a computing device such as a desktop computer, a laptop computer, a notebook, a smartphone, or the like, or can be any device that can be integrated. In order to perform the initial design function using such a first terminal 10, it must of course be equipped with software capable of performing the initial design function.

Using this first terminal 10, the patient is able to generate at least one post-operative 3D image of the desired surgery.

The second terminal 20 is similarly a computing device that can be utilized by the surgeon to finalize the surgical plan, which may be a computing device such as a desktop computer, a laptop computer, a notebook, a smartphone, or the like, or can be any device that can be integrated. In order to perform the finalization function using such a second terminal 20, it must of course be equipped with software capable of performing the finalization function. Using this second terminal 20, the surgeon may determine the final postoperative 3D image by checking the post-operative 3D image desired by the patient.

In this case, the software that performs the initial design function and the software that performs the final design function can be separate software, or it can be provided as one integrated software with different usage rights for different users.

The server 30 is one or more computer systems or computer software, implemented as a standalone server or a network server, configured at least a plurality of first terminals 10 and second terminals 20 to transmit/receive a corresponding data via a wired/wireless network.

Here, a network server refers to a computer system and computer software (network server program) that is connected with a subordinate device capable of communicating with other network servers via a computer network such as a private intranet or the internet to receive a request to perform a task, perform the task, and provide the result of the performance.

However, in addition to such a network server program, it is to be understood as a broad concept that includes a series of applications operating on the network server and, in some cases, various databases established internally or externally. In particular, in order for such a server 30 to perform the function of producing a prosthesis according to the present disclosure, it must of course be equipped with software capable of processing and sending/receiving data according to the present disclosure.

Further, in accordance with the present disclosure, a database may be accessible or equipped to store, and the data included in the database may comprise, for example, a patient medical imaging data, a 3D image based on the patient medical imaging data, at least one modified 3D image corresponding to an initially designed surgical plan by the patient, and a final 3D image corresponding to a finalized surgical plan by the surgeon.

In addition, the system 1 may further include a third terminal 40 that can be utilized to generate and transmit a 3D image based on the patient medical image data to the server 30. Similar to the first terminal 10 or second terminal 20 above, the third terminal 40 may be as a computing device, such as a desktop computer, a laptop computer, a notebook, a smartphone, or the like, or any other device that may be or may be integrated with a computing device. In order to perform the 3D image generation function using such a third terminal 40, it must of course be equipped with software capable of performing the 3D image generation function, which may be modified by the first terminal 10 or the second terminal 20. Such software may be implemented as conventional software, such as conventional CAD.

In this case, the software that can perform the 3D image generation function may be a software that exists separately from the software that can perform the initial design function and the software that can perform the final design function described above, or it may be provided as one integrated software with different usage rights depending on the user.

As described below, this third terminal 40 may also provide an interface that allows the user to check and fabricate a design of the implant determined based on the patient medical imaging data, the 3D image, and the final 3D image.

Furthermore, as described above, a fourth terminal 50 may be defined, incorporating the functions of a server 30 for designing an implant according to a surgical plan and a third terminal 40 that provides an interface for generating a 3D image based on patient medical imaging data and/or enabling confirmation and fabrication of the determined implant design.

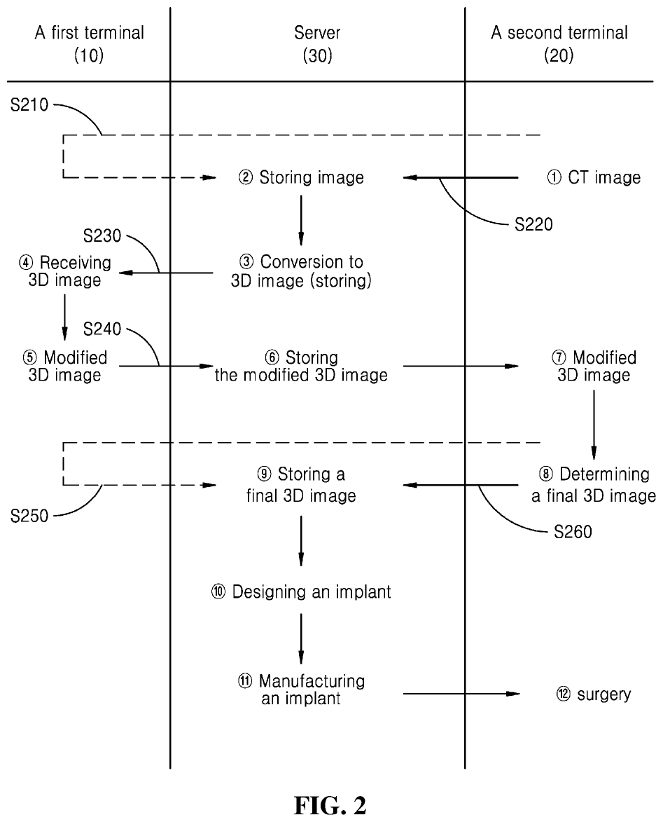

FIG. 2 is a flow diagram of a method for fabricating a surgical implant according to one embodiment of the present disclosure, which may be executed on the system 1 as described above.

A method of fabricating a surgical implant according to the present disclosure, comprising (1) acquiring a 3D image based on data of a patient's medical image (refer to \textcircled{1} of FIG. 2) including at least a body part to be operated on (refer to \textcircled{2} and \textcircled{3} of FIG. 2).

In this case, the patient medical imaging data may be any imaging data, such as CT images, 3D scanning data, or the like, taken for the purpose of medical practice, that includes at least the surgical site, from which it is possible to acquire the implantation surface on which the implant is to be implanted before surgery, and the three-dimensional overall contour of the surgical site before and/or after surgery.

On the other hand, under the current medical law, in order for the server 30, which is a computing device equipped with software for designing an implant according to a surgical plan, to access the patient medical image data of a medical institution, the patient medical image data can be provided directly from the patient himself or a legitimate representative such as a spouse. If the proposed revised law is enacted, the server 30 may access the patient medical image data if the patient or the patient's representative requests that the medical record be transmitted to a third party designated by the patient or the patient's representative. Thus, as shown in S210 of FIG. 2, the patient medical image data may be provided to the server 30 directly through the patient's first terminal 10, or, as shown in S220 of FIG. 2, the patient medical image data may be transmitted by the medical institution to a designated address, fax, e-mail, location, etc. upon the patient's request.

The step of acquiring a 3D image based on the patient medical imaging data may include, for example, converting the CT image to a 3D image and receiving and/or storing the converted 3D image. The conversion to a 3D image may be performed as described above, by the server 30, or by a separately provided third terminal 40, followed, in some cases, by the reception and/or storage of the 3D image (or a data thereof) are processed.

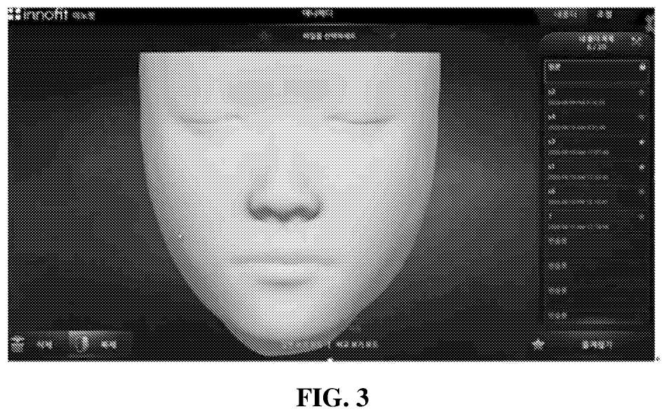

An example of a converted 3D image may be shown in FIG. 3. The converted 3D image according to the present disclosure shows an overall outline, for example, of a three-dimensional surgical part before surgery, as described above, and may be modified using a software of the first terminal 10.

Next, the method of fabricating a surgical implant according to the present disclosure may comprise (2) transmitting a 3D image (or a data thereof) from the server 30 to the first terminal 10 (see \textcircled{4} of FIG. 2), modifying the received 3D image by the first terminal 10, and obtaining and storing at least one modified 3D image by the server 30 (see \textcircled{6} of FIG. 2), that is corresponding to the surgical plan initially designed by the patient and transmitted from the first terminal 10 (see \textcircled{5} of FIG. 2). That is, the step of obtaining the modified 3D image may comprise, for example, modifying the 3D image and receiving and/or storing the modified 3D image, similar to the step of obtaining the 3D image.

Since S230 and S240 of FIG. 2 are transmissions and receipts about a patient medical image data once acquired between a patient and a server 30 as a third party other than a medical institution and a patient (e.g., an implant manufacturer or an implant design company), based on a prior agreement (e.g., an agreement to provide personal information including medical records), the modified 3D image may be provided to the server directly from the patient or a third party who is a legitimate representative of the patient (e.g., a spouse).

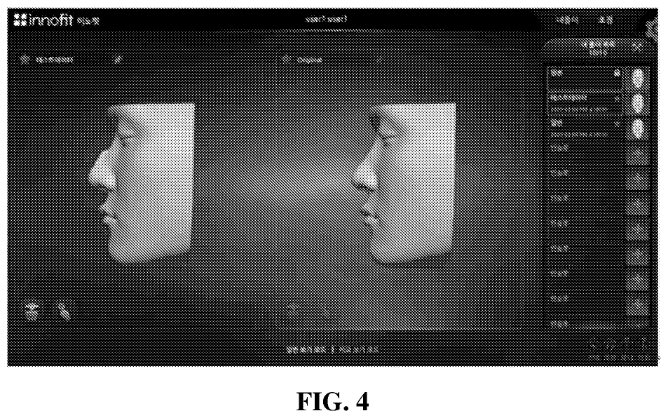

FIGS. 4 through 6 are drawings illustrating the process of modifying a 3D image, for example, in response to a patient's initial design of a surgical plan.

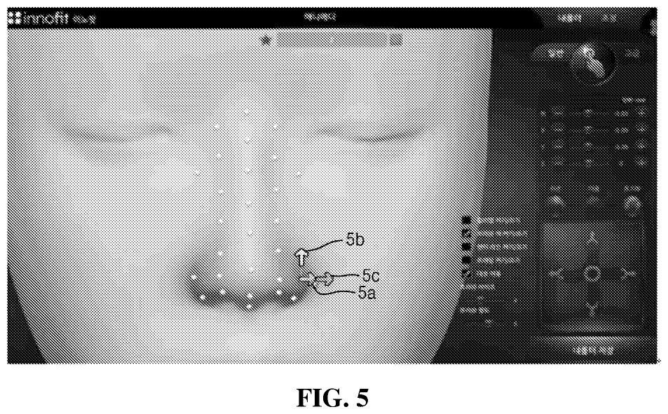

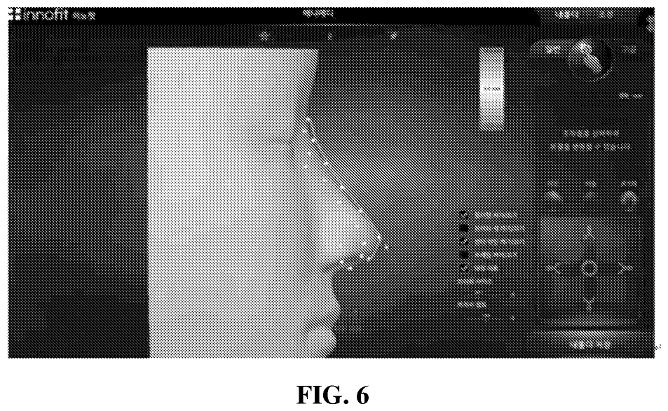

FIG. 4 shows a 3D image obtained based on the patient's medical imaging data (left) and a modified 3D image according to the patient's preferences (right), and FIG. 5 shows a modified 3D image generated by adjusting the shape of the tip of the nose and FIG. 6 shows a modified 3D image generated by adjusting the shape of the bridge of the nose.

In this case, the modified 3D image may be generated by clicking and dragging in a certain direction from a point at a certain location using the software according to the present disclosure on the first terminal 10, as shown in FIGS. 5 and 6.

For example, referring to FIG. 5, if the patient wishes to enlarge the tip of the nose, grabbing arrow 5a and pulling it in the direction of arrow 5c, i.e., outward (or grabbing arrow 5b and pulling it upward) will cause the areas in the vicinity (shown in the drawing) to adjust appropriately based on learning, such as machine learning, or a predefined formula to adjust to the natural shape.

In addition, numerical input can be used in conjunction with or instead of click-and-drag, as it may be difficult to make fine-tuning adjustments to a design with click-and-drag.

In addition, the method may further comprise the step of providing software to the first terminal 10, that capable of modifying the 3D image on the first terminal 10, prior to or at the time of transmitting the 3D image to the first terminal 10, as described above. In this case, the step of providing the software capable of modifying the 3D image may include, for example, providing the first terminal 10 with an address from which the software can be downloaded, downloaded and/or installed.

Next, the method of producing a surgical implant according to the present disclosure may comprise (3) transmitting the at least one modified 3D image to the second terminal 20 (see \textcircled{7} of FIG. 2) and acquiring from the second terminal 20 a final 3D image (see \textcircled{9} of FIG. 2) corresponding to the surgical plan finalized by the surgeon (see \textcircled{8} of FIG. 2). In this case, acquiring the final 3D image may include, for example, determining the final 3D image and receiving and/or storing the final 3D image, similar to the step of acquiring the 3D image.

On the other hand, in order for the server 30, which is a computing device equipped with software for designing an implant according to a surgical plan under the current medical law, to access the final 3D image corresponding to the surgical plan finalized by a surgeon of a medical institution, it is possible to be provided with the final 3D image directly from the patient or to access the final 3D image with the consent of the patient, as in the case of the patient medical image data. Thus, as shown at S250 in FIG. 2, the final 3D image may be provided to the server 30 via the patient's first terminal 10, or, as shown at S260 in FIG. 2, the final 3D image may be sent to a designated address, fax, e-mail, location, etc. by medical institution with the patient's consent.

Here, the 3D images modified and stored in the second terminal 20 according to the patient's preference may be one or more, and the modified images transmitted to the server and/or the second terminal 20 may be one or more, for example, three modified 3D images selected by the patient among a plurality of modified 3D images.

Furthermore, the determination of the final 3D image may further comprise selecting at least one of the received modified 3D images by the surgeon (surgeon) and re-modifying the selected modified 3D image based on evaluating whether the initially designed surgical plan is technically feasible and applicable to the patient in some cases for the selected at least one of the received modified 3D images. In order for the surgeon to make an expert judgment about the feasibility of the surgical plan corresponding to the modified 3D image incorporating the patient's input, the surgeon may rely on the patient's medical image data.

According to one embodiment of the present disclosure, in order to perform this more objectively and easily, software (hereinafter referred to as virtual surgery software) capable of performing the final design function mounted on the second terminal 20, as mentioned above, can be utilized.

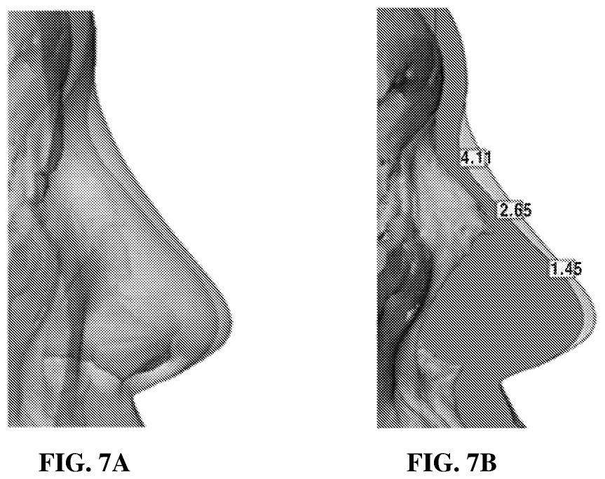

In this case, the virtual surgery software may display a screen such as FIG. 7A, wherein the anatomical interior based on the patient's medical imaging data, a 3D image, and a (re)modified 3D image, wherein the implantation surface of the surgical implant may be defined, are overlaid to allow a surgeon to make a final design. Using this, the modified 3D image may be revised at the overlapped view, or an overlapped view of the revised image may be shown at the virtual surgery software.

Additionally, or optionally, the virtual surgery software may display a screen such as FIG. 7B, wherein an anatomical interior based on patient medical imaging data (e.g., a CT image), a 3D image, and a (re)modified 3D image, wherein an implantation surface for the surgical implant (excluding cartilage) may be defined, are overlaid. Using this, the modified 3D image can be re-modified at the screen showing an overlapped shape, or the overlapping screen of the re-modified image can be shown. Meanwhile, the numerical shown in the FIGs may be the difference between the (re)modified 3D image and the 3D image, which may be the height of the implant.

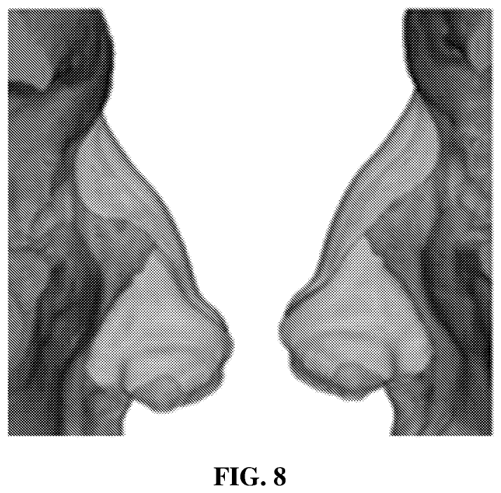

Additionally, or optionally, the virtual surgery software may display a screen as shown in FIG. 8, that shows an implantation surface which may be defined, where a surgical implant (including cartilage) is to be implanted, from an overlapped view of an anatomical interior based on patient medical imaging data (e.g., a CT image), a 3D image, and a (re)modified 3D image. Using this, the modified 3D image may be revised on the overlapped view, or an overlapped view reflecting the revision may be shown as a screen.

In this way, the surgeon may select one as a final 3D image among at least one of the modified 3D images transmitted to the second terminal 20 or may determine the final 3D image as re-modifying the selected modified 3D image. In this case, as in the above embodiments, when the surgical plan is finalized using the virtual surgery software mounted on the second terminal 20, the following steps of designing the implant may be performed simultaneously in whole or in part.

Furthermore, after the surgeon selects one as a final 3D image among at least one of the modified 3D images transmitted to the second terminal 20 or determines the final 3D image as re-modifying the selected modified 3D image, and prior to the hospital or the company contracted with the hospital to manufacture the implant, manufacture the implant based on the patient medical image data, the 3D image, and the final 3D image, the final 3D image may be confirmed by the patient's first terminal 10 and/or the surgeon's second terminal 20. Prior to receiving the confirmation for the final 3D image, the final 3D image may be complemented by at least one of the patient, the server, and the surgeon, and for this process, transmission and reception of the final 3D image may occur among them.

Next, the method of making a surgical implant according to the present disclosure may comprise the step of (4) designing the implant based on the patient medical imaging data, the 3D image, and the final 3D image (see \textcircled{10} of FIG. 2).

On the other hand, the height of the implant cannot be determined simply based on the difference between the postoperative 3D image and the preoperative 3D image, as the implant is inserted under the skin, over bone, cartilage, and/or mucous membranes, rather than placing on the preoperative skin. In order to minimize post-operative side effects, it is necessary to define the surface where the implant is placed (hereinafter, the placing surface) and to determine the height of the implant based on the difference between the post-operative 3D image and the pre-operative 3D image based on this defined placing surface where the implant is placed.

The placing surface for the implant can be derived, for example, using the method described in Patent Application No. 2018-0056637, filed by this Applicant on May 17, 2018, entitled “Nose Implant Manufacturing Method”. In this method, the geometry of the nasal cartilage, which is not visible in the CT image, is modeled by applying anatomical elements to the images that are visible in the CT image—nasal bone image and nasal cavity image—to define a placing surface on which the implant is to be placed. Furthermore, it is disclosed to model the inner shape, which is the placing surface of the nose implant, based on the modeled nasal cartilage.

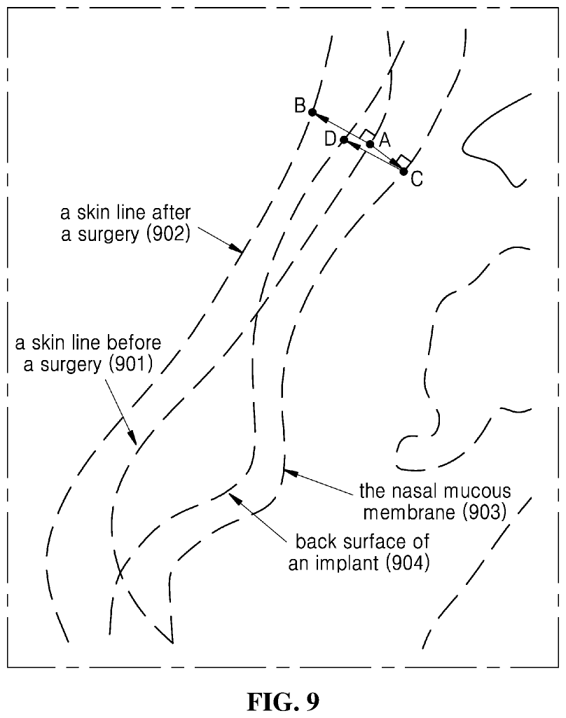

To determine the height of the implant based on the difference between the post-operative 3D image and the pre-operative 3D image with respect to the defined placing surface, FIG. 9 is shown.

Referring to FIG. 9, the dotted line labeled post-operative mucosa 903 may be seen as a placing surface for the implant, the dotted line labeled (virtual) post-operative skin 902 may be seen as a post-operative 3D image, and the dotted line labeled pre-operative skin 901 can be seen as a pre-operative 3D image.

FIG. 9 illustrates acquiring the height of the implant from the difference between the pre-operative skin and the (hypothetical) postoperative skin, for each cross-section perpendicular to the length of the nose, for example. Of course, the method for acquiring the difference may vary depending on how a point on the pre-operative skin is mapped to a point on the post-operative skin and how a point on the placing surface is mapped to these two points.

A method of acquiring the height of an implant according to the present disclosure first navigates from any point A on the preoperative skin 901 to the nearest point B on the (hypothetical) postoperative skin 902. Where A is a vector (arrow AB) from A to B, corresponding to the start of a perpendicular straight line from the pre-operative skin 901 line toward the post-operative skin 902 line.

Next, from any point A on the preoperative skin 901, the arrival point C of the perpendicular line to the mucosa 903 may be connected (arrow AC).

Finally, the point displaced from a point C above the mucosa 903 by a vector corresponding to arrow AB is defined as the height of the implant D (arrow CD).

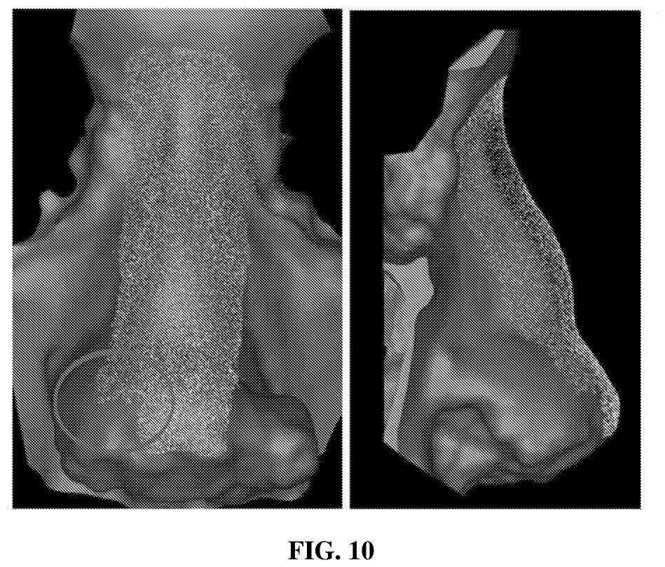

By thus determining the implant height D for each of the plurality of points placed on the preoperative skin 901 at predetermined appropriate intervals, for each of the perpendicular cross-sections along the length of the nose, a predetermined volume of all or at least some of the plurality of points for all or at least some of the plurality of cross-sections is achieved, as shown in FIG. 10.

Thus, this predetermined volume may correspond to the shape of the designed implant.

On the other hand, the shape of the implant designed by the above process may not be smooth, as shown in the circled portion of FIG. 10. In such a case, if the implant is manufactured as designed according to the present disclosure, the rough edges may cause injury to the surgeon during the operation or leave scars on the patient's surgical part.

In such cases, the outline of the implant may optionally be adjusted by deleting the part of the outline of the predetermined volume that has a point density below the predetermined standard, or by adjusting the width of the implant using the reference value set based on the accumulated data, or by free-curve modeling based on the skin shape after surgery.

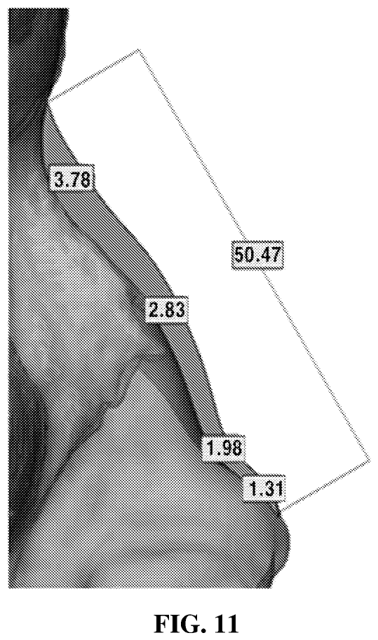

This completes the design of the implant, for example, as shown in FIG. 11, which is a drawing showing the final design of the implant according to the present disclosure.

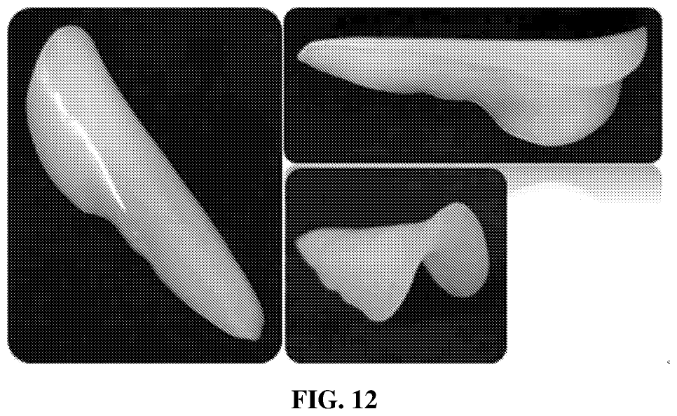

The method of fabricating a surgical implant according to the present disclosure further comprises the step of fabricating the implant designed according to the disclosure (see \textcircled{11} of FIG. 2), wherein the step of fabricating the designed implant may use at least one of 3D printing, cutting, injection, mold, or vacuum forming. This completes the fabrication of the implant, as shown in FIG. 12.

In order to fabricate the designed implant, as mentioned above, for example, the third terminal (not shown in Figure) may provide an interface that allows the user to view and fabricate the design of the implant determined based on the patient medical imaging data, the 3D image, and the final 3D image.

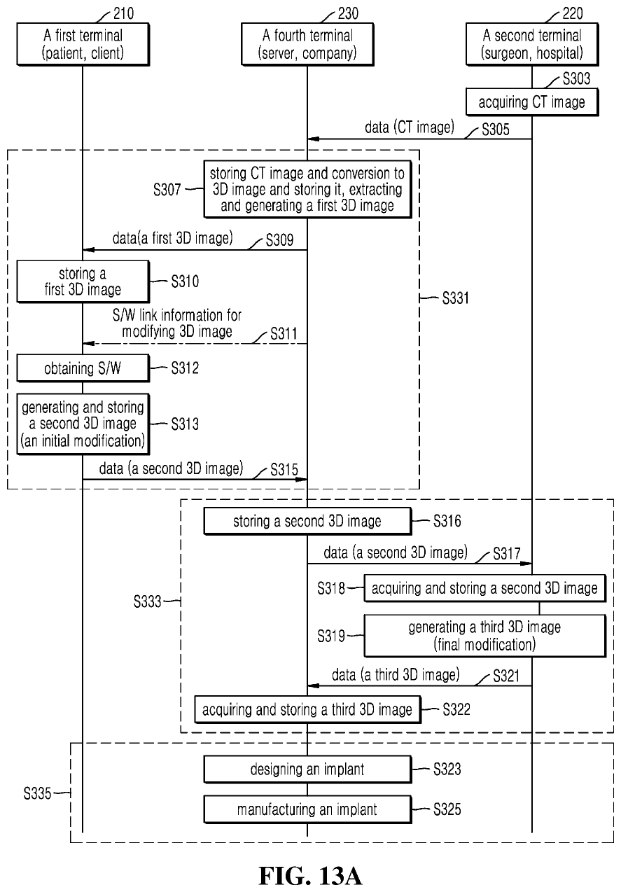

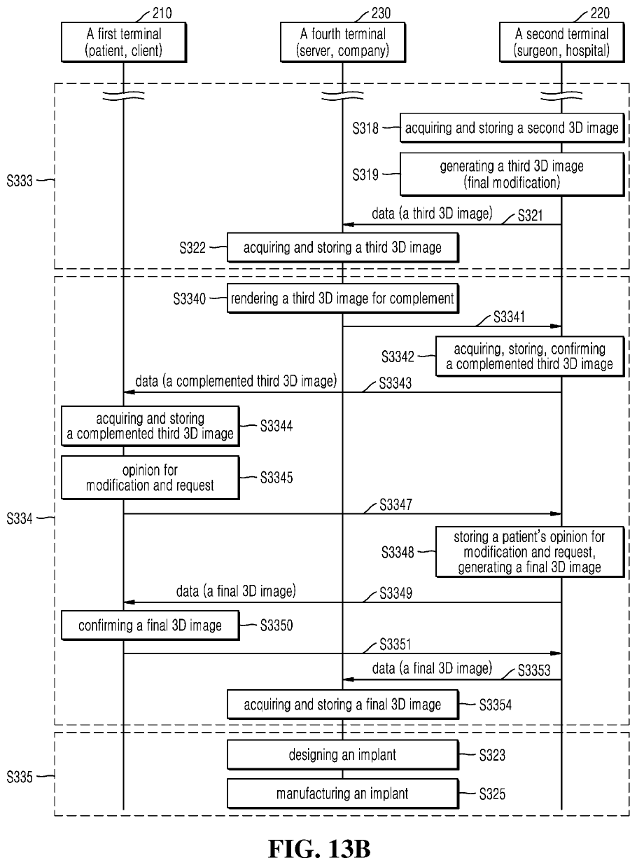

FIGS. 13A and 13B are each flow diagrams of a method of fabricating a surgical implant according to one embodiment of the present disclosure. FIGS. 13A and 13B are more specific illustrations of the method described with reference to FIG. 2, and the same or similarly applicable terms and steps are omitted from the description.

Referring to FIG. 13A, a method for producing a surgical implant according to the present disclosure comprises obtaining CT image S303 and transmitting CT image to a fourth terminal S305 as patient medical image data including at least a surgical part by a medical institution such as a hospital. Although described as a second terminal in the Figure, it refers to at least one computing device on which the present disclosure is executed in a medical institution such as a hospital, surgeon, or the like, and is not intended to limit the apparatus for acquiring and transmitting the CT image and/or the apparatus for acquiring the second 3D image and/or the apparatus for generating the third 3D image to be described later to occur on one physically identical device.

On the other hand, in order for the fourth terminal 230, which includes a server as a computing device equipped with software for designing an implant, according to a surgical plan, to access the patient medical image data of a medical institution, under the current medical law, the patient medical image data may be provided directly from the patient himself or herself, or the patient medical image data may be accessed with the consent of the patient himself or herself. Thus, the patient medical image data may be provided to the fourth terminal 230 via the patient's first terminal 210, as shown in S210 in FIG. 2, or may be transmitted to an address, fax, e-mail, designated location, etc. designated by the patient, by a medical institution with the patient's consent, as shown in S305 in FIG. 13A.

Specifically, step S331 receives data from the patient that corresponds to the initial design of the surgical plan.

At the fourth terminal 230, the CT image transmitted (S305) is stored S307, converted to a 3D image based on it, stored, and at least one first 3D image to be transmitted to the patient is extracted and generated (S307) and transmitted (S309) to the first terminal 210. Herein, the converted 3D image to be transmitted to the patient is named the first 3D image.

The first terminal 210 stores S310 the first 3D image transmitted S309 and, generates and stores S313 a modified first 3D image that is a second 3D image, using software capable of modifying the first 3D image, and transmits S315 it to the fourth terminal 230. For this purpose, an address from which this software can be downloaded is provided by the fourth terminal 230 S311 and the software is obtained from this address S312.

Specifically, the step S333 shows the process which receives a data from the surgeon that corresponds to the final design of the surgical plan.

The fourth terminal 230 stores the second 3D image transmitted S315 with the patient's input and transmits it S317 to the surgeon's second terminal 220. In addition, the fourth terminal 230 may generate a revised second 3D image with some design modifications to the received second 3D image as needed in the process of manufacturing the implant and transmit it to the second terminal 220.

The second terminal 220 stores S318 the second 3D image transmitted S317, modifies at least one second 3D image using software capable of modifying the second 3D image, generates and stores S319 a third 3D image, and transmits S321 the third 3D image to the fourth terminal 230.

On the other hand, in order for the fourth terminal 230, which includes a server as a computing device equipped with software for designing an implant in accordance with a surgical plan under the current medical law, to access the third 3D image corresponding to the surgical plan finalized by a surgeon of a medical institution, the third 3D image may be provided directly from the patient himself or herself, or the third 3D image may be accessed with the consent of the patient himself or herself. Accordingly, the patient medical image data may be provided to the fourth terminal 230 via the patient's first terminal 210, as shown in S210 in FIG. 2, or may be transmitted to an address, fax, e-mail, designated location, etc. designated by the patient, by the medical institution with the patient's consent, as shown in S321 in FIG. 13A.

The step of S335 designing S323 and fabricating S325 the implant may be performed on the fourth terminal 230, based on the third 3D image transmitted S321. This may be adopted as described with reference to \textcircled{10} and \textcircled{11} of FIG. 2.

According to embodiments, the step of S335 may be performed at the hospital or at a company, depending on the location of the server. For example, it may be performed at the second terminal 220 coupled to the server, or it may be performed at the fourth terminal 230, as the hospital request manufacturing to the fourth terminal 230.

FIG. 13B is a flowchart of a method of fabricating a surgical implant according to one embodiment of the present disclosure, which may further include the step of S334 confirming the final 3D image from a patient or/and a surgeon, between the step of S333 receiving data corresponding to a final design of a surgical plan, and the step of S335 designing and manufacturing an implant based thereon.

Referring to FIG. 13b for looking specifically at the step S334 confirming the third 3D image as a final 3D image, the acquired third 3D image is complemented and rendered S3340, and then the complemented third 3D image is transmitted to the second terminal 220 S3341 for confirmation by the surgeon and/or patient.

For example, the step of receiving a confirmation from the patient may occur between the first terminal 210 and the second terminal 220, starting with the second terminal 220 transmitting the acquired complemented third 3D image S3342 to the first terminal 210 S3343.

After acquiring and storing the complemented third 3D image (S3344), the first terminal 210 creates a revised opinion and request (S3345) and transmits it to the second terminal 220 (S3347). In this case, the revised opinion and request may be related to the patient's opinion for the complemented third 3D image compared to the first revised second 3D image on the first terminal 210. The patient may modify the 3D image on the first terminal 210 directly using software that allows modification of the 3D image, or may be written in text and/or pictures and stored and transmitted along with the third 3D image, or separately.

The second terminal 220 stores the transmitted patient's revisions and requests (S3348) and generates a final 3D image reflecting them and transmits it to the first terminal 210 (S3349).

Thereafter, the first terminal 210 may confirm the final 3D image (S3350) and transmit a confirmation message (S3351). In this case, the confirmation message may not include the final 3D image itself, but only the confirmation information. After receiving the confirmation, the second terminal 220 may transmit the final 3D image to the fourth terminal 230 (S3353).

On the other hand, in order for the fourth terminal 230, which includes a server as a computing device equipped with software for designing an implant according to a surgical plan under the current medical law, to access the final 3D image of a medical institution, the final 3D image may be provided directly from the patient himself or herself, or the final 3D image may be accessed by the fourth terminal 230 with the consent of the patient himself or herself. Thus, the patient medical image data may be provided to the fourth terminal 230 directly via the patient's first terminal 210, as shown in S210 in FIG. 2, or may be transmitted by the medical institution with the patient's consent to an address, fax, email, designated location, etc. designated by the patient, as shown in S3353 in FIG. 13B.

Similar to the previous embodiments, the fourth terminal 230 may obtain the final 3D image and perform implant design and fabrication based on it.

Alternatively, it is similar to the previous embodiments that the second terminal 220 obtains the final 3D image and performs the design and fabrication of the implant based on it, or request it to the fourth terminal 230 for fabrication.

In summary, according to the method of fabricating surgical implant of the present disclosure, the patient's opinion and the doctor's judgment are simultaneously applicable to the implant because the doctor is consulted based on the data designed by the patient himself or herself.

In addition, the method of fabricating surgical implant according to the present disclosure enables simulation of the surgical plan in which the implant is inserted before surgery, which can reduce surgical errors, thereby reducing side effects and improving patient satisfaction.

Furthermore, the method of fabricating a surgical implant according to the present disclosure eliminates the need for carving of the implant during surgery, thereby reducing costs, including surgical time and human costs.

In addition, if carving the implant is difficult to fit inside of patient's nose, the patient's internal tissues may be excised, which can be minimized according to the method of fabricating surgical implant of the present disclosure, thereby reducing the occurrence of excessive postoperative swelling and the postoperative side effects thereby.

In addition to the effects of the present disclosure as mentioned above, additional effects and features of the present disclosure will be clearly understood by those skilled in the art from the following description of the present disclosure.

In general, terms used herein, particularly in the claims (e.g., in the body of the claims), are intended to be generally “open-ended” terms (e.g., “including” is to be construed as “including but not limited to,” “having” is to be construed as “having at least as much as,” and “comprising” is to be construed as “including but not limited to”). If a specific number is intended for an introduced claim term, such intent is expressly stated in the claim, and in the absence of such a statement, such intent is understood not to exist.

Only certain features of the disclosure have been shown and described herein, and various modifications and changes may occur to those skilled in the art. It is therefore understood that the claims are intended to cover changes and modifications that fall within the spirit of the disclosure.

The executable instructions for performing the operations of the device for providing Surgical implants in accordance with the embodiments may be stored in CRM or other computer program products, which are configured to be executed by one or more processor and are not temporary, or may be stored in CRM or other computer program products, which are configured to be executed by one or more processor and are temporary. Also, the memory according to the embodiments may be used as a concept that includes a non-volatile memory, a flash memory, PROM, etc. as well as a volatile memory (for example, RAM, etc.).

The components of the device for providing Surgical implants in accordance with the embodiments described in FIGS. 1 to 13B may be configured by their separate hardware (for example, chip, hardware, circuit, device capable of performing communication, etc.), or may be configured by one hardware. At least one of the components of the device for providing surgical implants in accordance with the embodiments may be configured by one or more processors that may execute a program.

Terms used herein, such as ‘first’, ‘second’, ‘third’, and ‘fourth’ may be used to describe the various components according to the embodiments. However, the various components according to the embodiments are not limited to the above terms. These terms are only used to identify one component from another component. For example, the first terminal may be referred to as the second terminal, and vice versa, and this change should be made within the range that does not depart from the various embodiments described as above. The first terminal, the second terminal, the third terminal, and the fourth terminal are all terminals but are not interpreted as the same terminal unless explicitly specified on the context. For example, the first 3D image may be referred to as the second 3D image, or the third 3D image and vice versa, and this change should be made within the range that does not depart from the various embodiments described as above. The first 3D image, the second 3D image, and the third 3D image are all images but are not interpreted as the same image unless explicitly specified on the context.

What is claimed is:

1. A method for fabricating a surgical implant, based on a system for a first terminal for initially designing a surgical plan by a patient, a second terminal for finalizing a surgical plan by a surgeon, and a server for communicating with the first terminal and the second terminal and further designing an implant according to the surgical plan, the method comprising:
obtaining, by the server, a first 3D image based on a patient medical image data including at least one of a surgical part;
transmitting, by the server, the first 3D image to the first terminal to obtain at least one modified second 3D image corresponding to the surgical plan initially designed by the patient, from the first terminal;
transmitting, by the server, the at least one modified second 3D image to the second terminal to obtain a final third 3D image corresponding to the surgical plan designed by the surgeon, from said second terminal; and
designing, by said server, the implant based on the patient medical image data, the first 3D image, the second 3D image, and/or the third 3D image.

2. The method of claim 1,
wherein before or at a time of transmitting the first 3D image to the first terminal, further includes:
providing software capable of modifying the first 3D image to the first terminal, by the server, wherein the modified second 3D image is generated using the software.

3. The method of claim 2, the modified second 3D image is generated by at least one of a click-and-drag method and a numerical input method from a point at a particular location to a particular direction using the software.

4. The method of claim 1, wherein the designing, by said server, the implant based on the patient medical image data, the first 3D image, the second 3D image, and/or the third 3D image, further comprises: acquiring a placing surface for the implant to install on from the patient medical image data, and
determining a shape and height of the implant, based on a difference among the third 3D image, the patient medical image data, the first 3D image, and/or the second 3D image relative to the placing surface.

5. The method of claim 4, further comprising manufacturing the designed implant, by the server, wherein the manufacturing the designed implant may be performed by at least one of 3D printing, cutting, injection, mold, and vacuum forming.

6. A method for fabricating a surgical implant based on a system for a first terminal for initially designing a surgical plan by a patient, a second terminal for finalizing a surgical plan by a surgeon, and a server for communicating with the first terminal and the second terminal and further designing an implant according to the surgical plan, comprising:
acquiring, by the first terminal, a first 3D image based on patient medical image data, including at least a surgical part;
generating, by the first terminal, at least one modified second 3D image corresponding to the surgical plan initially designed by the patient based on the acquired first 3D image;
transmitting, by the first terminal, the at least one modified second 3D image to the server and/or the second terminal;
receiving, by the first terminal, a third 3D image that is a final 3D image corresponding to the surgical plan finalized by the surgeon based on the at least one modified second 3D image generated; and
confirming, by the first terminal, the third 3D image to allow the second terminal to design the implant based on the patient medical image data, the first 3D image, the second 3D image, and/or the third 3D image.

7. The method of claim 6, wherein the confirming, by the first terminal, the third 3D image to allow the second terminal comprises requesting designing the implant to the server, by the second terminal.

8. The method of claim 7, further comprising transmitting a message for confirming the third 3D image to the second terminal and/or the server, by the first terminal.

9. The method of claim 8, wherein the transmitting, by the first terminal, the confirmed message for the third 3D image comprises transmitting a patient's opinion based on comparing the second 3D image with the third 3D image.

10. The method of claim 6, further comprising: before the generating, by the first terminal, at least one modified second 3D image corresponding to the surgical plan initially designed by the patient, providing to the first terminal software capable of modifying the second 3D image.

11. The method of claim 10, the modified second 3D image is generated by at least one of a click-and-drag method and a numerical input method from a point at a particular location in a particular direction using the software.

12. A method for fabricating a surgical implant based on a system for a first terminal for initially designing a surgical plan by a patient, a second terminal for finalizing a surgical plan by a surgeon, and a server for communicating with the first terminal and the second terminal and further designing an implant according to the surgical plan, the method comprising:
acquiring a 3D image, by the first terminal, based on a patient's medical image data including at least a surgical site;
generating, by the first terminal, at least one modified 3D image corresponding to the surgical plan initially designed by the patient based on the acquired 3D image;
acquiring, by the first terminal, a final 3D image corresponding to the surgical plan finalized by the surgeon based on the at least one modified 3D image; and
confirming, by the first terminal, the final 3D image to allow the server to design the implant based on the patient medical image data, the 3D image, and the final 3D image.

13. The method of claim 12, further comprising: before the generating, by the first terminal, at least one modified 3D image corresponding to the surgical plan initially designed by the patient, providing to the first terminal software capable of modifying the 3D image, wherein the modified 3D image is generated using the software.

14. The method of claim 13, wherein the modified 3D image is generated by at least one of a click-and-drag method and a numerical input method from a point at a particular location in a particular direction using the software.
